(19) United States
(12) Patent Application Publication
(10) Pub. No.: US 2004/0028735 A1
(43) Pub. Date: Feb. 12, 2004

(54) Pharmaceutical formulation

(76) Inventor: Unchalee Kositprapa, Davie, FL (US)

Correspondence Address: Ted W. Whitlock, Esq., Intellectual Property Counsel, Andrx Corporation, 4955 Orange Dr, Ft. Lauderdale, FL, 33314, US

(21) Appl. No.: 10/634,321

(22) Filed: Aug. 4, 2003

Related U.S. Application Data
(63) Continuation-in-part of application No. 09/597,206, filed on Jun. 20, 2000, now Pat. No. 6,602,522.
Application No. 09/597,206 is a continuation-in-part of application No. 09/335,575, filed on Jun. 18, 1999, now Pat. No. 6,077,541.
Application No. 09/335,575 is a division of application No. 08/970,489, filed on Nov. 14, 1997, now Pat. No. 6,096,340.
Application No. 09/335,575 is a continuation-in-part of application No. 09/143,167, filed on Aug. 28, 1998, now Pat. No. 6,174,548.

(51) Int. Cl. A61K 9/22

(52) U.S. Cl. 424/468

(57) ABSTRACT

A pharmaceutical formulation for oral administration of an pharmaceutically active compound is described which includes a tablet core containing an uncoated granulation of a therapeutically effective amount of at least one pharmaceutically active ingredient, an optional surface active agent, an optional pharmaceutically acceptable alkaline agent and a combination of at least one water soluble binder and at least one water insoluble binder, whereby controlled release is achieved by way of the water soluble and water insoluble binders.

### CROSS-REFERENCE TO RELATED APPLICATION

This is a continuation-in-part of U.S. patent application Ser. No. 09/597,206, filed Jun. 20, 2000, which is a continuation-in-part of U.S. patent application Ser. No. 09/335,575, filed Jun. 18, 1999, which is a divisional of U.S. patent application Ser. No. 08/970,489, filed Nov. 14, 1997; and is a continuation-in part of U.S. patent application Ser. No. 09/143,167, filed Aug. 28, 1998.

### BACKGROUND OF THE INVENTION

The present invention relates to compositions and methods for stabilizing active ingredients in pharmaceutical formulations and/or otherwise protecting these actives from degradation by chemical components present in either an overcoating or overlayer that is in communication with such actives, or alternatively present in the environment in which such actives are intended to function. In particular, one embodiment of the present invention relates to a stable tablet formulation of a pharmaceutical compound or composition, and more particularly relates to a stable formulation for an acid-labile compound, e.g., a substituted benzamidazole, such as the proton pump inhibitor, omeprazole.

For example, it is well known that certain therapeutic compounds are sensitive to acidic conditions and can degrade or otherwise change after contact with an acid. The well-known compound, omeprazole, degrades and will not function in its intended manner when it contacts the acidic conditions of the stomach.

Historically, alkaline materials were added to a core of omeprazole to buffer or neutralize the environment, i.e., the acidic conditions of the stomach, to which the compound was exposed during use. Enteric coatings were later applied over the omeprazole core to prevent the acidic pH conditions of the stomach from contacting the omeprazole. Providing an enteric coating over the omeprazole core can be satisfactory if the product is administered within a short time after its manufacture. However, if the product is stored under ambient conditions, the acidic residue of the enteric coating can degrade the omeprazole active ingredient before it is administered to a patient. Similarly, other chemical components present in coating compositions may deleteriously affect the active ingredient(s) of a formulation especially when in communication with these coatings over an extended period of time.

To solve this problem, certain formulations in the prior art have used a separate layer or other barrier of a coating agent to coat a pellet core, one example of such a core comprising omeprazole and an alkaline material. These coated pellets are thereafter further coated with an additional final overlayer of enteric coating. This technique of providing a separate or second additional coating, i.e., a dual layer, as described in U.S. Pat. No. 4,786,505, can be disadvantageous in that it requires two separate coating steps in its manufacture. Thus, the length of the manufacturing process for the product and the resulting costs are increased.

The applicants have surprisingly discovered a novel formulation and method which (1) avoids the need to use a separate or dual coating layer to physically isolate actives from the detrimental effects of an overcoating, such as an acid-labile active ingredient (e.g. substituted benzamidazole such as omeprazole) from an enteric coating layer; (2) provides a means for manipulating or controlling bioavailability of the active ingredient by providing cohesiveness of the powdered ingredients upon tablet disintegration; and (3) provides a unique release mechanism for pharmaceutical formulations having pharmaceutically active compounds disposed within a tableted core.

In addition, the subject formulation can advantageously provide a tablet dosage form that is bioequivalent to a capsule dosage form of the same or substantially similar strength. The tablet dosage form can further be advantageous in that the manufacturing process can require fewer steps, e.g., eliminate the need for pellet formation and/or coating of those pellets, and there is no need for the additional expense of providing capsule shells.

### SUMMARY OF THE INVENTION

The present invention relates to compositions and methods for stabilizing active ingredients in pharmaceutical formulations and/or otherwise protecting these actives from degradation by chemical components present in either an overcoating or overlayer that is in communication with such actives, or alternatively present in the environment in which such actives are intended to function. In one particular embodiment, the present invention concerns a novel dosage form or formulation for an acid-labile compound, e.g., a substituted benzamidazole such as omeprazole. This particular embodiment of the invention involves the use of an enteric coating agent applied to a core of an active ingredient, such as omeprazole, and a particular binder as a suspension in a suitable solvent.

Other embodiments of the present invention include tablet formulations and methods favorable for stabilizing and/or protecting active compounds that may not be acid labile but rather require, as a function of the intended pharmaceutical/therapeutic objective, either a sustained, delayed, and/or otherwise controlled release in and/or after passage through, an acid environment. Alternative embodiments of the present invention provide tablet formulations and methods that render a protective sequestering of actives that may be degraded or otherwise pharmacologically compromised not by the presence of acid, but rather other deleterious substances present in any coatings that are in communication with such actives and/or present in the formulation's functional or storage environments.

Importantly, the subject invention further concerns a formulation that employs a unique combination of a water-soluble and a water insoluble binder that surprisingly lends certain advantages to the pharmacokinetics and the stability of the active ingredient.

In a preferred embodiment, the subject formulation is an oral, controlled release pharmaceutical composition comprising a controlled release compressed tablet core made from a granulation comprising:

a) a therapeutically effective amount of at least one pharmaceutically active ingredient,

b) an optional surface active agent,

c) an optional pharmaceutically acceptable alkaline agent, and

d) at least one water soluble binder and at least one water insoluble binder;

wherein the controlled release is unexpectedly achieved by way of the combined water soluble and water insoluble binders. A further embodiment of the present invention includes a single layer of coating deposited on the core, most preferably an enteric coating agent.

Accordingly, it is an object of this invention to provide a pharmaceutical dosage formulation for a pharmaceutically active compound that is 1) protected from degradation by chemical components present in either an overcoating or overlayer that is in communication with such actives, or alternatively present in the environment in which such actives are intended to function; 2) stable upon prolonged storage; 3) stable when administered to a patient; and 4) is capable of providing the desired therapeutic effect by way of a sustained, delayed, and/or otherwise controlled release of the active.

It is yet a further object of this invention to provide a pharmaceutical dosage formulation for a pharmaceutically active ingredient, such as an acid-labile compound (e.g., a substituted benzamidazole as further exemplified by omeprazole) which is stable upon prolonged storage, is stable when administered to a patient, and is capable of providing the desired therapeutic effect. It is also an object of this invention to provide a tablet dosage form for a pharmaceutically active ingredient, such as an acid-labile compound (e.g., a substituted benzamidazole as further exemplified by omeprazole), which is bioequivalent to beaded capsule dosage forms which have an additional intermediate layer of an inert coating. It is also an object of this invention to provide a tablet dosage form for a pharmaceutically active ingredient, such as an acid-labile compound (e.g., a substituted benzamidazole as further exemplified by omeprazole) which is bioequivalent to beaded capsule dosage forms which have an additional intermediate layer of an inert coating material. It is a further object of this invention to provide a pharmaceutical dosage form for a pharmaceutically active ingredient, such as an acid-labile compound (e.g., a substituted benzamidazole as further exemplified by omeprazole) which is bioequivalent to dosage forms comprising a multiparticulate drug delivery system.

Yet another object of this invention is to provide a stable dosage form for a pharmaceutically active ingredient, such as an acid-labile compound (e.g., a substituted benzamidazole as further exemplified by omeprazole), which may be produced without the need for an intermediate coating layer that separates the tablet core from the enteric coating layer.

These and other objects of the invention will become apparent from a review of the appended specification.

### DETAILED DESCRIPTION OF THE INVENTION

The controlled release pharmaceutical composition of the subject invention is designed for oral administration and is preferably directed to a compressed tablet core formed from an uncoated controlled release granulation. The granulation is preferably comprised of a therapeutically effective amount of at least one pharmaceutically active ingredient, an optional surface-active agent, a filler, an optional pharmaceutically acceptable alkaline agent depending upon the active's sensitivity to acid, and a binder combination comprised of at least one water insoluble binder and at least one soluble binder. An enteric coating may be applied over the core as provided for below.

In one particular embodiment of the present invention, the formulation is preferably based on a compressed tablet core formed from a granulation that comprises an acid-labile compound as an active ingredient, e.g., a substituted benzamidazole such as omeprazole, an optional surface-active agent, a filler, a pharmaceutically acceptable alkaline material, and a binder.

The granulation core can comprise from about 5 to about 70 wt % and, preferably, can comprise about 10 to about 30 wt % of active ingredient. The formulation is advantageously adapted for use with a variety of pharmaceutically active compounds including analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-histamines, anti-hypertensive agents, anti-malarials, anti-migraine agents, anti-muscarinic agents, anti-neoplastic agents, erectile dysfunction improvement agents, hydantoins, immunosuppressants, anti-protozoal agents, anti-thyroid agents, anxiolytic agents, sedatives, hypnotics, neuroleptics, beta blockers, cardiac inotropic agents, corticosteroids, diuretics, anti-parkinsonian agents, gastro-intestinal agents, histamine receptor antagonists, keratolytics, anti-lipemic agents, anti-anginal agents, cox-2 inhibitors, leucotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, or mixtures thereof.

In one preferable embodiment, the invention is directed to acid-labile active ingredients, preferably a substituted benzamidazole. Substituted benzamidazoles are commonly known in the art and include, but are not limited to, proton pump inhibitors, e.g., omeprazole, lansoprazole, pantoprazole, perprazole, and the like, as well as pharmaceutically acceptable salts, isomers, or derivatives thereof.

Examples of analgesics include, but are not limited to salicylic acid, indomethacin, ibuprofen, fenoprofen, oxaprozin, meclofenamate, mefanamic acid, naproxen, naproxen sodium, flubiprofen, indoprofen, ketoprofen, piroxicam, diclofenac, etodolac, ketorolac, or pharmaceutically acceptable salts, isomers or derivatives thereof.

Examples of anti-convulsants include but are not limited to hydantoins (e.g. phenytoin), such as clonazepam, carbamazepam, valproic acid or pharmaceutically acceptable salts, isomers or derivatives thereof.

Examples of anti-diabetics include but are not limited to biguanide, meglitinide, sulfonylurea, thiazolidinedione, or pharmaceutically acceptable salts, isomers or derivatives thereof.

Examples of antilipemics include but are not limited to bile acid sequestrant, HMG-CoA reductase inhibitor, fibrate, fibric acid derivative, or pharmaceutically acceptable salts, isomers or derivatives thereof.

Examples of diuretics include but are not limited to carbonic anhydrase inhibitor, thiazide, potassium sparing diuretic or pharmaceutically acceptable salts, isomers or derivatives thereof.

Examples of anti-histamines include but are not limited to loratadine, fexofenadine, certirizine, or pharmaceutically acceptable salts, isomers or derivatives thereof.

Examples of anti-psychotics include but are not limited to is benzodiazepines, anti-anxiety agents, antidepressants, monamine oxidase inhibitors, selective serotonin reuptake inhibitors, tricyclic antidepressants, antimanic agents, antipanic agents, phenothiazines, chlorpromazines, triflupromazines, thioridazines, trifluoperazines, barbiturates, phenobarbitals, amobarbitals, pentobarbitals or pharmaceutically acceptable salts, isomers or derivatives thereof.

The surface-active agent is optional and can be any pharmaceutically acceptable, non-toxic surfactant, e.g., polysorbate 80 (Tween 80), or the like. The surface-active agent may be present at a level of up to about 5 wt % and, preferably, from about 0.20 to about 2.0 wt %, based on the total weight of the granulation.

The alkaline material is optional for non-acid labile compounds and can be sodium, potassium, calcium, magnesium or aluminum salts of phosphoric acid, carbonic acid, or citric acid, or can be aluminum/magnesium compounds such as Al₂O₃.6MgO.CO₂.12H₂O, (Mg₆Al₂(OH₁₋₆CO₃.4H₂O), or MgO.Al₂O₃.2SiO₂.nH₂O where n is a whole integer of 2 or more. Alternatively, the alkaline material can be lysine or arginine, or can be an antacid such as aluminum hydroxide, calcium hydroxide, magnesium hydroxide, or magnesium oxide. The alkaline agent is preferably provided at about 10 to about 80 wt % based on the total weight of the granulation, and would be understood by those of ordinary skill in the art to depend on the relative strength of the alkaline material. For example, arginine is typically utilized in the formulation from about 10 to about 60 wt %, and is preferably formulated at about 30 to about 55 wt %.

The binders can be any pharmaceutically acceptable combination of non-toxic water soluble and water insoluble binders such as the following water-soluble polymers, e.g., polyvinyl alcohol, polyvinylpyrrolidone, methylcellulose, hydroxypropyl cellulose, hydroxymethyl cellulose, and the following water-insoluble polymers, e.g., a polymethacrylic acid copolymer such as Eudragit NE30D. Eudragit NE30D is commercially available as a 30% aqueous dispersion. Preferably, the subject formulation comprises the unique combination of both a water-soluble and water-insoluble binder up to about 10 wt % in an aqueous medium such as water, or as an aqueous dispersion. More preferably, the binder combination is provided from about 0.25 to 7.5 wt % based on the total weight of the granulation.

A filler can also be used as a granulation substrate. As well understood in the art, sugars such as lactose, dextrose, sucrose, maltose, or microcrystalline cellulose or the like can be used as fillers in the granulation composition. The filler preferably can be provided from about 20 to 50 wt %, and more preferably about 25 to 40 wt % based on the total weight of the granulation.

A tablet disintegrant, e.g., cornstarch, potato starch, croscarmelose sodium, Crospovidone, or sodium starch glycolate, can also be included in the subject formulation in an effective amount. An effective amount of tablet disintegrant can be provided at about 1 to about 15 wt %, preferably from about 3 to about 8 wt %, based on the total weight of the granulation.

The enteric coating agent, if applied, can be any pharmaceutically acceptable material which resists acid up to a pH of about 5.0 or higher. Preferably, the enteric coating ingredient is selected from cellulose acetate phthalate, hydroxypropylmethyl cellulose phthalate, polyvinyl acetate phthalate, carboxymethylethylcellulose, Eudragit NE30D, Eudragit L (polymethacrylic acid:methylmethacrylate, 1:1 ratio; MW (No. Av. 135,000—USP Type A)) or Eudragit S (polymethacrylic acid:methylmethacrylate, 1:2 ratio MW (No. Av. 135,000—USP Type B)) and, most preferably, can be a mixture thereof. For example, Eudragit L100-55 is a 100% polymer solids product while the Eudragit L30-55 product is a 30% w/w aqueous dispersion of the polymer.

The enteric coating agent can also include an inert processing aid in an amount from about 10 to about 50 wt %, and preferably from about 20 to about 40 wt %, based on the total weight of the acid resisting component and the inert processing aid. The inert processing aid can include finely divided forms of talc, silicon dioxide, magnesium stearate or the like.

Typical solvents which may be used to apply the acid resisting component-inert processing aid mixture include isopropyl alcohol, acetone, methylene chloride, the like. Generally the acid-resistant component/inert processing aid mixture will be employed from about 5 to about 20 wt % based on the total weight of the solvent and the acid-resistant component/inert processing aid.

The enteric coating can optionally comprise a plasticizer. Suitable plasticizers for use in the enteric coating include acetyl triethyl citrate, dibutyl phthalate, tributyl citrate, triethyl citrate, acetyl tributyl citrate, propylene glycol, triacetin, polyethylene glycol and diethyl phthalate. The amount of plasticizer can vary, but will typically be present in amounts up to about 40% w/w based upon the weight of acid resisting component of the coating. More preferably, the plasticizer can be provided at about 10-20% w/w based upon the weight of the acid resisting component.

The granulation is preferably formed by combining the alkaline agent (in the case of acid-labile compounds), the active ingredient, (e.g., omeprazole, ketoprofen, etc.) to the surface active agent, and the water soluble and water insoluble binder combination with an acceptable solvent. An acceptable solvent can be any low viscosity medium such as water, isopropyl alcohol, acetone, ethanol or the like. Use of solvents such as water usually requires, a solvent weight about three times the weight of the dry components of the coating composition.

After the granulation is formed and dried, the granulation can be tableted by standard procedures as accepted in the art. The tablets can then be directly coated with the enteric coating agent, employing standard coating procedures. A color-imparting agent may be added to the enteric coating agent mixture or a rapidly dissolving seal coat containing color may be coated over the enteric coating agent layer provided that the seal coat is compatible with and does not affect the dissolution of the enteric coating layer. The rapidly dissolving seal coat can, for example, comprise Opadry pink which comprises approximately 91 wt % hydroxypropyl methylcellulose (E-6), color, and about 9 wt % polyethylene glycol applied as a 8-15% w/w solution in purified water. In addition, the color may be provided as “Chromateric” which is available from Crompton & Knowles. This product contains water, talc, TiO₂, triethyl citrate, propylene glycol, synthetic red iron oxide, potassium sorbate, xanthan gum, sodium citrate, and synthetic yellow iron oxide. If desired, conventional sugar based seal coats can be used which contain FDA-certified dyes.

### EXAMPLES

### Example 1

A. Granulation.

A granulation comprising an acid-labile active ingredient (the “active ingredient granule”) is formed in a fluid bed coater using a top spray granulation-forming suspension having micronized active ingredient, e.g., omeprazole. 5% w/w polyvinyl pyrrolidone; 2% w/w L-arginine; 0.5% w/w polysorbate 80; 0.4% w/w polymethacrylic acid copolymer, e.g., Eudragit NE30D; and purified water. The suspension is sprayed onto a mixture of microcrystalline cellulose and 92% w/w of the total amount of L-arginine. The formulation for making the granulation using omeprazole as the active ingredient has the following composition.

| | Wt. | % |
|---|---|---|
| Povidone, USP (Plasdone K30) | 97.6 g | 5.37 |
| Microcrystalline cellulose (Avicel PH101) | 465.7 g | 25.62 |
| L-arginine, USP/FCC | 731.7 g | 40.25 |
| Omeprazole, (USP, micronized)¹ | 487.8 g | 26.84 |
| Polysorbate 80 | 9.7 g | 0.53 |
| Methylmethacrylic acid (Eudragit NE30D) | 25.2 g | 1.39 |

¹95% of the particles exhibit a particle size of less than 15 microns

B. Tableting.

The granulation is formed into tablets comprising 20 mg of active ingredient hereinafter, (“the omeprazole tablet”) by standard tableting procedures. Specifically, the granules comprising omeprazole were mixed with Crospovidone and microcrystalline cellulose (Avicel PH101), then with glyceryl monostearate, in the following amounts:

| | |
|---|---|
| Omeprazole granules | 160.7 g |
| Glyceryl monostearate (EASTMAN 600P) | 13.5 g |
| Crospovidone | 79.6 g |
| Avicel PH101 | 16.2 g |

Conventional tableting procedures were performed to form the tablet as follows:

| | |
|---|---|
| Tableting tools: | 0.2812″ |
| Target weight: | 124 mg/tablet |
| Target hardness: | 7 Kp |

C. Enteric Coating.

An enteric coating was applied to prepare enteric coated tablets as follows:

| | |
|---|---|
| Omeprazole tablets (prepared as in Ex. 1, Sect. B., above) | 105.6 g |
| Hydroxypropyl methylcellulose phthalate 50 | 12.0 g |
| Talc | 1.2 g |
| Acetyl tributyl citrate | 1.2 g |
| Acetone | 80.0 g |
| Isopropyl alcohol | 80.0 g |

The solid coating materials were dissolved in the acetone and isopropyl alcohol and this suspension was coated onto the omeprazole tablets using a perforated pan.

### Example 2

A. Granulation.

A granulation comprising an acid labile active ingredient is formed in fluid bed coater using a top spray granulation-forming suspension containing micronized active ingredient, e.g., omeprazole; 5%w/w of the total amount of L-arginine; polyvinyl pyrrolidone; sodium lauryl sulfate; a polymethacrylic acid copolymer, e.g., Eudragit NE30D; and purified water. This suspension is sprayed onto a mixture of microcrystalline cellulose, 95%w/w of the total amount of L-arginine and sodium starch glycolate. The formulation for making the granulation has the following composition:

| | Wt. | % |
|---|---|---|
| Eudragit NE30D | 33.0 g | 1.81 |
| Povidone, USP (Plasdone K30) | 98.0 g | 5.38 |
| Sodium lauryl sulfate, NF/USP | 6.0 g | 0.33 |
| Microcrystalline cellulose (Avicel PH102) | 463.0 g | 25.44 |
| L-arginine, USP/FCC | 732.0 g | 40.22 |
| Omeprazole, (USP, micronized)¹ | 488.0 g | 26.82 |
| Purified water, USP | 1600.0 g | |

¹95% of the particles exhibit a particle size of less than 15 microns

B. Tableting.

The granulation is formed into tablets containing 20 mg of omeprazole by first mixing the omeprazole granules with crospovidone and microcrystalline cellulose (Alvicel PH101), then with glyceryl monostearate, as follows:

| | |
|---|---|
| Omeprazole granules | 160.7 g |
| Glyceryl monostearate (EASTMAN 600P) | 13.5 g |
| Crospovidone | 79.6 g |
| Avicel PH101 | 16.2 g |

Conventional tableting procedures were carried out to obtain tablets as follows:

| | |
|---|---|
| Tableting tools: | 0.2812″ |
| Target weight: | 124 mg/tablet |
| Target hardness: | 7 Kp |

C. Enteric Coating.

An enteric coating was applied to prepare enteric-coated tablets as follows:

| | |
|---|---|
| Omeprazole tablets (as prepared in Ex. 1, Sect. B., above) | 124.0 g |
| Hydroxypropyl methylcellulose phthalate 55 | 14.7 g |
| Talc | 4.2 g |
| Acetyl tributyl citrate | 2.9 g |
| Acetone | 148.0 g |
| Isopropyl alcohol | 148.0 g |

The solid coating materials were dissolved in the acetone and isopropyl alcohol and this suspension was coated onto the omeprazole tablets using a perforated pan.

D. Seal Coat.

A seal coat was applied to the enteric coated tablets as follows:

| | |
|---|---|
| Enteric coated tablet | 146.0 g |
| Opadry II pink | 4.5 g |
| Water | 450.0 g |

The seal coat was applied onto the enteric coated omeprazole tablets using a perforated pan coater.

### Example 3

A. Granulation.

A granulation comprising an acid labile active ingredient was formed in fluid bed coater using a top spray granulation-forming suspension containing micronized omeprazole; 2.0% w/w of the total amount of L-arginine; polyvinyl pyrrolidone; polysorbate 80; and a polymethyl methacrylic acid copolymer, e.g., Eudragit NE30D. The suspension is sprayed onto a mixture of microcrystalline cellulose and 95.0% w/w of the total amount of L-arginine. The formulation for making the granulation has the following composition:

| | Wt. | % |
|---|---|---|
| Povidone, USP (Plasdone K30) | 4.0 g | 4.77 |
| Polysorbate 80 (Tween 80) | 0.4 g | 0.48 |
| Eudragit NE30D | 0.4 g | 0.48 |
| L-arginine, USP/FCC | 40.0 g | 47.73 |
| Omeprazole, (USP, micronized)² | 20.0 g | 23.87 |
| Microcrystalline cellulose (Avicel PH102) | 19.0 g | 22.67 |

²95% of the particles exhibit a particle size of less than 15 microns

B. Tableting.

The granulation is formed into tablets containing 20 mg of omeprazole by first mixing the omeprazole granules with Crospovidone XL and Avicel PH102, then with glyceryl monostearate, as follows:

| | | |
|---|---|---|
| Omeprazole granules | 74.6 | mg |
| Glyceryl monostearate (EASTMAN 600P) | 9.0 | mg |
| Crospovidone XL | 11.8 | mg |
| Microcrystalline cellulose (Avicel PH102) | 79.6 | mg |

Tableting was performed using conventional tableting procedure to obtain tablets as follows:

| | |
|---|---|
| Tableting tools: | 0.3125″ |
| Target weight: | 175 mg/tab |
| Target hardness: | 7 Kp |

C. Enteric Coating.

An enteric coating was applied to prepare enteric coated tablets as follows:

| | | |
|---|---|---|
| Omeprazole tablets (as prepared in B., above) | 135.0 | mg |
| Eudragit L30D-55 | 14.0 | mg |
| Color (Chromateric ®) | 7.0 | mg |

The solid coating materials were dispersed in the water and this mixture was coated onto the omeprazole tablets using a perforated pan.

### Example 4

A. Granulation.

A granulation comprising an acid labile active ingredient is formed in fluid bed coater using a top spray granulation-forning suspension containing micronized active ingredient, e.g., omeprazole; 2.0% w/w of the total amount of L-arginine; polyvinyl pyrrolidone; polymethylmethacrylic acid copolymer, e.g., Eudragit NE30D; and purified water. The suspension is sprayed onto a mixture of microcrystalline cellulose and 95.0% w/w of the total amount of L-arginine. The formulation for making the granulation has the following composition, in mg/tablet:

| | Wt. | | % |
|---|---|---|---|
| Povidone, USP (Plasdone K30) | 2.0 | g | 5.42 |
| Eudragit NE30D | 0.16 | g | 0.43 |
| Polysorbate 80 | 0.2 | g | 0.54 |
| L-arginine, USP/FCC | 15.01 | g | 40.65 |
| Omeprazole, (USP, micronized)³ | 10.0 | g | 27.09 |
| Microcrystalline cellulose (Avicel PH101) | 9.55 | g | 25.87 |

³95% of the particles exhibit a particle size of less than 15 micros

B. Tableting.

The granulation is tabletted into tablets containing 10 mg of active ingredient, e.g., omeprazole, by first mixing the omeprazole granules with sodium starch glycolatye and Avicel PH102, then with glyceryl monostearate:

| | | |
|---|---|---|
| Omeprazole granules | 36.9 | mg |
| Glyceryl monostearate (EASTMAN 600P) | 8.75 | mg |
| Sodium starch glycolate | 10.5 | mg |
| Microcrystalline cellulose (Avicel PH102) | 118.9 | mg |

tableting was performed by conventional procedures with the following specifications:

| | |
|---|---|
| Tableting tools: | 0.3125″ |
| Target weight: | 175 mg/tablet |
| Target hardness: | 7 Kp |

C. Enteric Coating.

The tablets were coated with the same enteric coating that was applied to the tablets in Example 3, above.

### Example 5

A. Granulation.

A granulation containing omeprazole is formed in fluid bed coater using a top spray granulation forming suspension containing micronized omeprazole, 2.0%w/w of the total amount of L-arginine, polyvinyl pyrrolidone, polysorbate 80, polymethacrylic acid copolymer, and purified water which is sprayed onto a mixture of microcrystalline cellulose, and 95.0%w/w of the total amount of L-arginine. The formulation for making the granulation has the following composition in mg/tablet:

| | Wt. | | % |
|---|---|---|---|
| Povidone, USP (Plasdone K30) | 8.00 | mg | 5.42 |
| Polymethacrylic a copolymer | 0.62 | mg | 0.42 |
| Polysorbate 80 | 0.80 | mg | 0.54 |
| L-arginine, USP/FCC | 60.0 | mg | 40.65 |
| Omeprazole, (USP, micronized)⁴ | 40.0 | mg | 27.10 |
| Microcrystalline cellulose | 38.18 | mg | 25.87 |
| Purified water, USP | n/a | | |

⁴95% of the particles exhibit a particle size of less than 15 micros

B. Tableting.

The granulation is tabletted into tablets containing 20 mg of omeprazole by first mixing the omeprazole granules with glyceryl monostearate:

| | | |
|---|---|---|
| Omeprazole granules | 147.6 | mg |
| Glyceryl monostearate (EASTMAN 600P) | 7.4.1 | mg |
| Tableting tools: | 0.2812″ | |
| Target weight: | 155 | mg/tab |
| Target hardness: | 7 | Kp |

C. Enteric Coating.

The tablets were coated with the same enteric coating that was applied to the tablets in Example 1.

### Example 6

A. Granulation.

The granulation of Example 1 was prepared and tabletted into tablets containing 20.0 mg of omeprazole. These tablets were coated as follows:

B. Enteric Coating.

An enteric coating was applied to prepare enteric-coated tablets as follows:

| | | |
|---|---|---|
| Omeprazole tablets (prepared above) | 124.00 | mg |
| Eudragit L30D-55 | 17.00 | mg |
| 1 M NaOH (pH adjuster to pH 5.0) qs | | na |
| Acetyl tributyl citrate | 1.70 | mg |
| Talc | 3.80 | mg |
| Polysorbate 80 | 1.50 | mg |
| Purified water qs | | na |

The coating polymer was diluted with water and the other coating materials were added. This mixture was coated onto the omeprazole tablets using a perforated pan; a seal coat was applied using the procedure of Example 2.

### Example 7

A. Granulation.

A granulation comprising an antidiabetic active ingredient is formed in fluid bed coater using a top spray granulation-forming suspension containing active ingredient, e.g., glipizide; polyvinyl pyrrolidone; sodium lauryl sulfate; a polymethacrylic acid copolymer, e.g., Eudragit NE30D; and purified water. This suspension is sprayed onto a mixture of microcrystalline cellulose, 95%w/w of the total amount of sodium starch glycolate. The formulation for making the granulation has the following composition:

| | Wt. | | % |
|---|---|---|---|
| Eudragit NE30D | 33.0 | g | 1.81 |
| Povidone, USP (Plasdone K30) | 98.0 | g | 5.38 |
| Sodium lauryl sulfate, NF/USP | 6.0 | g | 0.33 |
| Microcrystalline cellulose (Avicel PH102) | 1439.0 | g | 79.07 |
| Glipizide | 244.0 | g | 13.41 |
| Purified water, USP | 1600.0 | g | |

B. Tableting.

The granulation is formed into tablets containing 10 mg of glipizide by first mixing the glipizide granules with crospovidone and microcrystalline cellulose (Alvicel PH101), then with glyceryl monostearate, as follows:

| | | |
|---|---|---|
| glipizide granules | 160.7 | g |
| Glyceryl monostearate (EASTMAN 600P) | 13.5 | g |
| Crospovidone | 79.6 | g |
| Avicel PH101 | 16.2 | g |

Conventional tableting procedures were carried out to obtain tablets as follows:

| | |
|---|---|
| Tableting tools: | 0.2812″ |
| Target weight: | 124 mg/tablet |
| Target hardness: | 7 Kp |

### Example 8

A. Granulation.

A granulation comprising an antilipemic active ingredient is formed in fluid bed coater using a top spray granulation-forming suspension containing micronized active ingredient, e.g., lovastatin; 5%w/w of the total amount of L-arginine; polyvinyl pyrrolidone; sodium lauryl sulfate; a polymethacrylic acid copolymer, e.g., Eudragit NE30D; and purified water. This suspension is sprayed onto a mixture of microcrystalline cellulose, 95%w/w of the total amount of L-arginine and sodium starch glycolate. The formulation for making the granulation has the following composition:

| | Wt. | | % |
|---|---|---|---|
| Eudragit NE30D | 33.0 | g | 1.81 |
| Povidone, USP (Plasdone K30) | 98.0 | g | 5.38 |
| Sodium lauryl sulfate, NF/USP | 6.0 | g | 0.33 |
| Microcrystalline cellulose (Avicel PH102) | 1257.0 | g | 69.07 |
| L-arginine, USP/FCC | 182.0 | g | 10.00 |
| Lovastatin | 244.0 | g | 13.41 |
| Purified water, USP | 1600.0 | g | |

B. Tableting.

The granulation is formed into tablets containing 10 mg of lovastatin by first mixing the lovastatin granules with crospovidone and microcrystalline cellulose (Alvicel PH101), then with glyceryl monostearate, as follows:

| | | |
|---|---|---|
| Lovastatin granules | 160.7 | g |
| Glyceryl monostearate (EASTMAN 600P) | 13.5 | g |
| Crospovidone | 79.6 | g |
| Avicel PH101 | 16.2 | g |

Conventional tableting procedures were carried out to obtain tablets as follows:

| | |
|---|---|
| Tableting tools: | 0.2812″ |
| Target weight: | 124 mg/tablet |
| Target hardness: | 7 Kp |

### Example 9

A. Granulation.

A granulation comprising an antihypertensive is formed in fluid bed coater using a top spray granulation-forming suspension containing active ingredient, e.g., felodipine; 5%w/w of the total amount of polyvinyl pyrrolidone; sodium lauryl sulfate; a polymethacrylic acid copolymer, e.g., Eudragit NE30D; and purified water. This suspension is sprayed onto a mixture of microcrystalline cellulose, 95%w/w of the total amount of sodium starch glycolate. The formulation for making the granulation has the following composition:

| | Wt. | | % |
|---|---|---|---|
| Eudragit NE30D | 33.0 | g | 1.81 |
| Povidone, USP (Plasdone K30) | 98.0 | g | 5.38 |
| Sodium lauryl sulfate, NF/USP | 6.0 | g | 0.33 |
| Microcrystalline cellulose (Avicel PH102) | 1439.0 | g | 79.07 |
| Felodipine | 244.0 | g | 13.41 |
| Purified water, USP | 1600.0 | g | |

B. Tableting.

The granulation is formed into tablets containing 10 mg of felodipine by first mixing the felodipine granules with crospovidone and microcrystalline cellulose (Alvicel PH101), then with glyceryl monostearate, as follows:

| | | |
|---|---|---|
| Felodipine granules | 160.7 | g |
| Glyceryl monostearate (EASTMAN 600P) | 13.5 | g |
| Crospovidone | 79.6 | g |
| Avicel PH101 | 16.2 | g |

Conventional tableting procedures were carried out to obtain tablets as follows:

| | |
|---|---|
| Tableting tools: | 0.2812″ |
| Target weight: | 124 mg/tablet |
| Target hardness: | 7 Kp |

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof that do not depart from the spirit and scope of the invention.

What is claimed is:

1. An oral controlled release pharmaceutical composition having a controlled release core, said core comprising:
a) a therapeutically effective amount of at least one pharmaceutically active ingredient;
b) an optional surface active agent;
c) an optional pharmaceutically acceptable alkaline agent; and
d) at least one water soluble binder and at least one water insoluble binder;
wherein the controlled release is achieved by way of the water soluble and water insoluble binders.

2. The composition of claim 1, further comprising a single layer of coating on said core, said coating comprising an enteric coating agent.

3. The composition of claim 1 wherein the pharmaceutically active ingredient is selected from analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-histamines, anti-hypertensive agents, anti-malarials, anti-migraine agents, anti-muscarinic agents, anti-neoplastic agents, erectile dysfunction improvement agents, hydantoins, immunosuppressants, anti-protozoal agents, anti-thyroid agents, anxiolytic agents, sedatives, hypnotics, neuroleptics, beta blockers, cardiac inotropic agents, corticosteroids, diuretics, anti-parkinsonian agents, gastro-intestinal agents, histamine receptor antagonists, keratolytics, anti-lipemic agents, anti-anginal agents, cox-2 inhibitors, leucotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, or mixtures thereof.

4. The composition of claim 1 wherein the optional alkaline material is selected from lysine, arginine, sodium, potassium, calcium, magnesium or aluminum salts of phosphoric acid, carbonic acid or citric acid.

5. The composition of claim 1 wherein the optional alkaline material is selected from an aluminum hydroxide, calcium hydroxide, magnesium hydroxide, or magnesium oxide.

6. The composition of claim 1, wherein the water-insoluble binder is a polymethacrylic acid copolymer.

7. The composition of claim 1 wherein the enteric coating comprises a component selected from cellulose acetate phthalate, hydroxypropylmethyl cellulose phthalate, polyvinyl acetate phthalate, carboxymethylethylcellulose, or co-polymerized methacrylic acid/methacrylic acid methyl esters.

8. The composition of claim 2 wherein the enteric coating further comprises an inert processing aid.

9. The composition of claim 2 wherein the enteric coating further comprises from 10 to 80 wt % by weight of the coating of an inert processing aid.

10. The composition of claim 1 wherein the surface-active agent is sodium lauryl sulfate.

11. The composition of claim 3 wherein the pharmaceutically active ingredient is an analgesic, anti-inflammatory agent, anti-coagulant, anti-psychotic, anti-epileptic, diuretic, anti-lipemics or pharmaceutically acceptable salts, isomers or derivatives thereof.

12. The composition of claim 3 wherein said analgesic is salicylic acid, indomethacin, ibuprofen, fenoprofen, oxaprozin, meclofenamate, mefanamic acid, naproxen, naproxen sodium, flubiprofen, indoprofen, ketoprofen, piroxicam, diclofenac, etodolac, ketorolac, or pharmaceutically acceptable salts, isomers or derivatives thereof.

13. The composition of claim 3 wherein said anti-convulsant is phenytoin, clonazepam, carbamazepam, valproic acid or pharmaceutically acceptable salts, isomers or derivatives thereof.

14. The composition of claim 3 wherein said antidiabetic is a biguanide, meglitinide, sulfonylurea, thiazolidinedione, or pharmaceutically acceptable salts, isomers or derivatives thereof.

15. The composition of claim 3 wherein said antilipemic is bile acid sequestrant, HMG-CoA reductase inhibitor, fibrate, fibric acid derivative, or pharmaceutically acceptable salts, isomers or derivatives thereof.

16. The composition of claim 3 wherein said diuretic is carbonic anhydrase inhibitor, thiazide, potassium sparing diuretic or pharmaceutically acceptable salts, isomers or derivatives thereof.

17. The composition of claim 3 wherein said anti-histamine is loratadine, fexofenadine, certirizine, or pharmaceutically acceptable salts, isomers or derivatives thereof.

18. The composition of claim 3 wherein said anti-psychotic is benzodiazepine, an anti-anxiety agent, an antidepressant, a monamine oxidase inhibitor, a selective serotonin reuptake inhibitor, a tricyclic antidepressant, an antimanic agent, an antipanic agent, phenothiazine, or pharmaceutically acceptable salts, isomers or derivatives thereof.

19. The composition of claim 18 wherein said anti-psychotic is chlorpromazine, triflupromazine, thioridazine, trifluoperazine, a barbiturate, phenobarbital, amobarbital, pentobarbital or pharmaceutically acceptable salts, isomers or derivatives thereof.

20. A oral controlled release pharmaceutical composition having a controlled release core, said core consisting essentially of:
a therapeutically effective amount of a pharmaceutically active ingredient, an optional surface active agent, an optional pharmaceutically acceptable alkaline agent, at least one water soluble binder and at least one water insoluble binder;
wherein said controlled release is achieved through the use of said water soluble and water insoluble binders.

21. A method for manipulating bioavailability of a pharmaceutical dosage formulation comprising a core having powdered components and a coating, said method comprising the step of providing at least one water-insoluble binder and at least one water soluble binder in the core to control cohesiveness of powdered core components upon disintegration of the core.

22. The method of claim 21, wherein the water-insoluble binder is a polymethacrylic acid copolymer.
